(19) United States
(12) Patent Application Publication
(10) Pub. No.: US 2009/0305223 A1
(43) Pub. Date: Dec. 10, 2009

(54) METHOD AND APPARATUS FOR EVALUATIING INTERACTIONS IN ISOLATED TISSUE

(76) Inventor: Irving WEINBERG, Bethesda, MD (US)

Correspondence Address: BARNES & THORNBURG LLP, 750-17TH STREET NW, SUITE 900, WASHINGTON, DC, 20006-4675, US

(21) Appl. No.: 12/476,368

(22) Filed: Jun. 2, 2009

Related U.S. Application Data
(60) Provisional application No. 61/059,559, filed on Jun. 6, 2008.

(51) Int. Cl. A01N 1/02; C12M 3/00; C12Q 1/02

(52) U.S. Cl. 435/1.2; 435/287.1; 435/29

(57) ABSTRACT

An apparatus for evaluating the effects of stimuli on tissue includes an environment for holding at least a sample of the tissue from a living person. The apparatus also has a first source connected to the environment for delivering nutrients to the tissue to maintain the tissue in a viable condition for more than one week. A second source is connected to the environment for delivering a biologically active agent to the tissue. The biologically active agent is capable of inducing carcinogenesis in the tissue when metabolized. An effluent outlet is connected to the environment to remove effluent from the environment.

### CROSS-REFERENCE TO RELATED APPLICATION(S)

This is a U.S. Non-Provisional Patent Application that relies for priority on U.S. Provisional Patent Application Ser. No. 61/059,559, filed on Jun. 6, 2008, the contents of which are incorporated herein by reference.

### FIELD OF THE INVENTION

Without limitation, the present invention is directed to an apparatus and a method for research and development of drug therapies using isolated tissues. In particular, the invention provides a research apparatus and method to facilitate research for the development of therapies to be applied to diseased tissue.

### DESCRIPTION OF RELATED ART

When developing therapies for various ailments, researchers follow generally a multi-phase approach.

This multi-phase approach may encompass, for example, a researcher's development of pharmaceuticals that block certain biochemical processes within cancer cells.

In some prior art examples with respect to the study of cancers, researchers initially acquire immortalized cancer cells to establish a tissue culture test sample.

Immortalized cancer cells generally refer to cancer cells that have been isolated for study and are maintained in a viable condition. Immortalized cancer cells often are available through tissue culture collections, as should be known to those skilled in the art.

As a general rule, cancerous tissues are not typically excised from patients and utilized for research. In most cases, cancerous tissues are treated as biological waste and are destroyed, pursuant to strict guidelines. However, certain cell lines have been preserved for research. Those cell lines include immortalized cells lines.

Once a sample of immortalized cancers cells are obtained, researchers often apply biochemical blockers (or other biologically active agents) to the tissue culture and observe if the cells react in a favorable or unfavorable manner. In the case of a biochemical blocker, the researcher may seek to determine if the candidate pharmaceutical blocker being added to the tissue culture is effective in inhibiting an undesired biochemical reaction. These experiments occur outside of the living organism: experiments outside of a living organism are referred to as “in vitro” experiments.

If the researcher finds that a particular blocker has a desired effect on the immortalized cancer cells, the researcher typically will advance the research from culture studies to experiments involving living animals, such as mice. These studies are conducted within a living organism: experiments in living organisms as referred to as “in vivo” experiments.

Typically, when the researcher experiments on a living organism, diseased cells are first introduced into the living organism (i.e., the mouse). This process is denoted a xenograft study. Subsequently, a candidate pharmaceutical is introduced into the same mouse, and the growth of the xenograft site is measured in order to assess effectiveness of the candidate pharmaceutical.

Upon completion of successful testing in the living organism, the researcher may proceed to testing the pharmaceutical in humans.

One of the drawbacks to the approach outlined above lies in the fact that the researcher typically works with only one type of cell, whether in culture (in vitro) or in the xenograft model (in vivo).

Study of a single cell type may not provide the researcher with a comprehensive understanding of the interactions of the cell type with surrounding, healthy cells and the applied biologically-active agent. Certain diseases are believed to arise as the result of the interplay of varied types of cells. For example, the tissue organization field theory (“TOFT”) of carcinogenesis and metastasis (TOFT) presumes that normal cells may be influenced to become cancerous as a result of their neighbors. As evidence of this theory, W W Barclay et al., in Endocrinology 146:13-18, 2005, showed that studying epithelial-stromal interactions reveals distinct inductive abilities of stromal cells from benign prostatic hyperplasia and prostate cancer. In other words, when human prostate epithelium cells where placed with normal supporting prostate cells (“stroma”), the epithelium cells did not become cancerous. However, when the same cells were placed in abnormal immune-compromised tissue, the epithelium cells did become cancerous.

There is further evidence suggesting that the influence of neighboring cells can cause human epithelial cells to transform into carcinomas (i.e., become cancerous). For example, D J Flint and C H Knight (in J Mammary Gland Biol and Neoplasia, 1997; 2(1): 41-48), posit that stromal breast cells, under the influence of circulating Growth Hormone, elaborate insulin growth factor (type 1), which increases proliferation rates of epithelial cells.

In order to study the mechanism by which cells interact with one another and generate cancerous cells, it is desirable to measure the interaction of several tissue types. More specifically, it is desirable to measure interaction between different tissue types in a milieu that is as close to human physiological conditions as possible.

As noted above, one particular area of interest is the study of breast cancer genesis, treatment, and remission.

Human females undergo different hormonal processes than many other animals. For example, the phenomenon of menopause, in which females forego ovulation for a significant portion of their lives, is rare outside of human species (see, e.g., Daryl P. Shanley, Rebecca Sear, Ruth Mace, Thomas B. L. Kirkwood, “Testing evolutionary theories of menopause,” Proceedings of the Royal Society: Biological Sciences (10.1098/rspb.2007.1028) (2007)).

Because the human female hormonal process differs from many other animals, it becomes difficult to study cancers in other animals and derive general principles for cancer development in human females, especially with respect to breast cancer. In addition, the study of breast cancers is hampered by various proscriptions against testing in human beings. These difficulties present a challenge to researchers.

Before proceeding to a summary of the invention, additional information about art related to the present invention is provided below in summary format.

As should be appreciated by those skilled in the art, Langendorff has described at least one method for keeping an animal heart alive after the heart is removed from the body. U.S. Pat. No. 7,045,279 provides an overview of this methodology, because the '279 Patent invokes essentially the Langendorff preparation.

U.S. Pat. No. 6,743,181 describes a system and method for measuring ventricular function in an isolated perfused heart for species including rats, mice, and guinea pigs.

U.S. Pat. No. 7,223,413 discusses modalities for arresting, preventing, or preserving. Specifically, the '413 Patent concerns methods of preserving an organ for open-heart surgery or other interventions.

U.S. Pat. No. 5,985,539 discusses methods for preserving organs (“reconstructing animal organs”) and perfusing the organ through its vascular system via a network of cavitized structures.

A review of additional related art suggests that at least some research has focused on isolating perfused breast tissue, which has been accomplished for specific animals (e.g., cows and goats) but not humans, to look at physiology (e.g., vasodilation).

With respect to studies of cow udders, reference is made to an article at http://www.pubmedcentral.nih.gov/articlerender.fcgi?artid=2290480, by Zeitlin and Esraghi. In that article, the authors state: “Amongst the vasodilator agents that Prosser et al. considered, parathyroid hormone-related protein, insulin-like growth factor-I (IGF-1), prostacyclin, nitric oxide and endothelin have been shown to be produced by the mammary gland. Prosser et al. (1996) also thought that since mammary tissue contained tissue kallikrein (Peeters et al. 1976), the kinins should also be considered as potentially important mammary vasodilators.” This article, however, does not sufficiently address the relationship between the ductal tissues of the breast and the surrounding supportive tissues (stroma), which is critical in studies of carcinogenesis.

In other related art, a discussion of the role of circulating IGF is given. One article on this subject is: C Diorio et al., “Insulin-like Growth Factor-I, IGF-Binding Protein-3, and Mammographic Breast Density,” Cancer Epidemiology, Biomarkers, and Prevention 14(5):1065-1073, 2005.

In still other related art, effects of steroidal and non-steroidal antiphlogistic drugs on eicosanoid synthesis in irritated skin has been studied, including studies with an isolated, perfused bovine udder. BÄUMER W, Kietzmann M., Journal of Pharmacy and Pharmacology. 2001 ;53:743-747.

The literature also suggests that skin from human breast tissues may be kept alive after removal during surgery. Reference is made to the following article on this point: R K Winkelmann, W Mitchell Sams Jr and Joan H King, “Contraction of Isolated Human Breast Cutaneous Vascular Smooth Muscle,” Journal of Investigative Dermatology (1973) 60, 297-300; doi:10.1111/1523-1747.ep12723096, which article may be found at http://www.nature.com/jid/journal/v60/n5/abs/5617719a.html.

As a result of one or more of the considerations listed above, it is desirable to develop a human model for cancer research, particularly breast cancer research, in which cells of many types in the breast are examined in as close to their normal milieu as possible. Optimally, human subjects in such a research study would undergo repeated sampling, before and after administration of candidate pharmaceuticals. Obviously, this is not possible because of ethical and procedural considerations.

Therefore, at least due to the prohibitions and cautions against in vivo research, there has developed a need for in vitro methodologies that may assist with pharmaceutical development.

### SUMMARY OF THE INVENTION

It is, therefore, one aspect of the present invention to offer an apparatus and method that facilitates evaluation of interactions in isolated tissues, including vital organs.

It is another aspect of the invention to provide an apparatus and a method that permits testing of chemical compounds, including pharmaceuticals, on tissue maintained in a viable state in vitro.

Still another aspect of the invention concerns a method and an apparatus which provide a platform for evaluating candidate interventions on a body part (typically the breast), in which epithelial cells are in a milieu that is close to the natural state.

Specifically, the present invention concerns an apparatus and a method where a multitude of different, closely-related tissue types are kept alive in an in vitro environment so that biological mechanisms may be studied.

One further aspect of the present invention is the provision of a method and an apparatus that addresses important procedural and ethical considerations.

In one contemplated embodiment, the invention is directed to an apparatus and method for the study and treatment of breast cancers. As should be apparent, however, the invention is not limited solely to the study of cancers but may be applied to any of a number of aliments and diseases.

To accomplish one or more of the objectives of the invention, an isolated animal breast is perfused after removal. In the case of non-human animals, the breast (udder) is removed after the animal has been slaughtered. In the case of humans, the breast or breast tissue may be removed in the course of mastectomy or lumpectomy. In the course of human mastectomy, normal and/or abnormal breast tissue may be removed, in the case where the breast being removed contains cancer. Alternatively, the breast tissue may be normal, in the case where the breast being removed is performed as a result of prophylactic mastectomy. The human breast could also be removed after the death of a patient, which may occur as a part of heart surgery.

For purposes of the invention, a perfusion system is provided to keep the breast alive.

For purposes of the invention, the effect of various hormonal and/or chemical factors within the perfusate is assessed with invasive and non-invasive methods. The invasive methods include, but are not limited to, biopsy and/or surgery of the isolated breast. The non-invasive methods include, but are not limited to, techniques typically employed in diagnostic imaging of the breast, including MRI, ultrasound, x-ray mammography, nuclear medicine and positron emission mammography, optical and infrared imaging. These imaging techniques may be performed with or without contrast material administered through the perfusate, and can be repeated as needed. In at least one contemplated embodiment, the invention incorporates noninvasive diagnostic methods and components that do not require imaging, for example, they could examine electric and/or electromagnetic fields around the breast.

A specific application of the invention lies in the administration of radioactively-tagged fluoro-deoxyglucose for PET imaging during compression of the isolated perfused breast, with or without high resolution contrast-enhanced MRI or x-ray imaging, in the presence of drugs designed to antagonize hormonal factors (e.g., IGF). For example, Genentech has an IGF antagonist drug (US PTO 7071300). Pfizer has pegvisomant (“somavert”), a GH antagonist. The rationale for using a glucose analog to examine the effects of IGF are based in part on the work of Mepham (T B Mepham, “The Development of Ideas on the Role of Glucose in Regulating Milk Secretion,” Aust. J. Agric. Res. 44:509-22, 1993).

Another specific application is the use of the invention to improve methods of detecting breast cancer using chemical assays or non-invasive diagnostic methods (whether together or in combination). It is anticipated that this approach may be potentially achieved by deriving formulas to be used in correcting functional imaging methods for factors that can be derived from biochemical or anatomic measurements (e.g., x-ray density). These relationships may be reasonably assumed to exist based in part on the work by DiOrio mentioned above.

The effect of interventions on the isolated perfused breast can be assessed using the above techniques. These interventions may include administration of chemical substances via the perfusate, or methods that interact with the isolated perfused breast in other ways, for example, via radiation administration to some (or all) of the isolated perfused breast.

The invention is applicable to organs other than the breast, for example, prostates.

Note that the use of the invention could lead to better drug development and understanding of dosing for existing and new drugs. The formulas for improving and combining imaging modalities and investigations described above could be used to improve selection of patients for, and administration of, drugs and methods of preventing and treating cancers. Note that the invention could lead to development of diagnostic tests combining non-invasive modalities and test administration of drugs, in a manner similar to cardiac stress tests.

Note that the isolated preparation could remain vital for a long period, as has been done for the heart (example: http://www.pulsus.com/ccc2004/abs/a482.htm). This would be helpful for studies of long-term effects of drugs or noxious agents.

Additional information about this invention is provided below in summary format. This additional information provides specific details at least in connection with the study of breast cancer. As should be apparent, however, the invention is not limited solely to the study of cancers but may be applied to any of a number of aliments and diseases.

### BRIEF DESCRIPTION OF THE DRAWINGS

The invention is described in connection with one or more illustrations appended hereto, in which:

FIG. 1 is a schematic illustration of a first embodiment of an apparatus according to the present invention;

FIG. 2 is a schematic illustration of a second embodiment of an apparatus according to the present invention;

FIG. 3 is a schematic illustration of a third embodiment of an apparatus according to the present invention;

FIG. 4 is a schematic illustration of a fourth embodiment of an apparatus according to the present invention;

FIG. 5 is a flow diagram, illustrating one method contemplated by the present invention; and

FIG. 6 is a flow diagram, illustrating a second method contemplated by the present invention, where this second method is an extension of the first method illustrated in FIG. 5.

### DETAILED DESCRIPTION OF EMBODIMENT(S) OF THE INVENTION

The invention is described in connection with one or more embodiments. The discussion of specific embodiments is not intended to be limiting of the invention. To the contrary, the exemplary embodiment(s) are intended to illustrate the breadth and scope of the invention. The invention is intended to encompass equivalents and variations on the embodiment(s) discussed, as should be appreciated by those skilled in the art.

It is noted further that the present invention is discussed in connection with cancer, specifically breast cancer. The invention, however, is not intended to be limited solely to cancers and/or breast cancer. To the contrary, the invention is intended to provide broad support for apparatuses and methods that permit researchers, doctors, and others to develop and test a wide variety of stimuli on in vitro tissues, as will be made apparent from the discussion that follows.

As a preliminary matter and for purposes of completeness, articles and literature discussed in this specification are incorporated herein by reference.

In one contemplated embodiment, the invention includes the isolation of a breast (preferably human), with the delivery of nutrients to the breast (see, e.g., A M Ehinger and M Kietzmann, in “Tissue Distribution of Oxacillin and Amplicillin in the Isolated Perfused Bovine Udder”, Journal of Veterinary Medicine Series A 47(3), 157-168, 2000).

In this contemplated embodiment, the breast is removed surgically, and major vessels of one or more breast segments are cannulated. Physiological fluids (e.g., Tyrode's solution with heparin, glucose, and blood gases) are infused via the cannulated vessels. The physiological fluids maintain the excised breast tissue in a living state.

As should be understood by those skilled in the art, isolated breast tissue is available from cadavers (recently deceased) or from surgeries performed on patients undergoing mastectomies, whether prophylactic, or therapeutic, and whether segmental or total. Alternatively, the tissue may be available from mammoplasty procedures or from tissue culture collections.

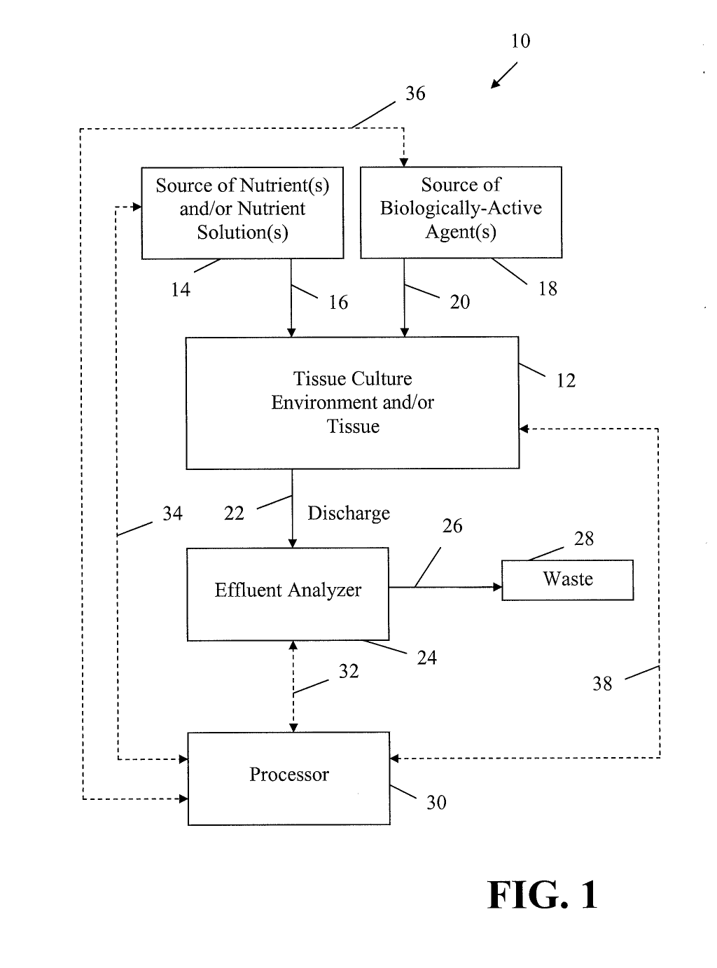

Reference is now made to FIG. 1, which illustrates a first embodiment contemplated to fall within the scope of the present invention.

FIG. 1 shows an apparatus for evaluating the effects of stimuli on tissue. The system is referred to herein as a tissue culture system 10. The tissue culture system 10 includes a tissue environment 12 into which the tissue sample is placed. The tissue environment 12 is anticipated to be a closed environment that maintains the tissue in sterile conditions.

For purposes of discussion, the tissue placed in the tissue environment may be a small portion of tissue, such as a tissue sample, or may be as large as an entire organ. The exact size and type of tissue is not particularly important for practice of the present invention. It is contemplated that the tissue sample may be on the order of only a few cells thick, where microscopic analysis of the tissue is desired or required. In other contemplated experiments, the entire organ may be required so that interactions of a wide variety of cells may be studied.

In still other contemplated experiments, it is envisioned that the tissue under study might also include portions of surrounding tissue from the original host. Under certain circumstances, it may be desirable to study the biological interactions between a plurality of tissues rather than focusing on an individual tissue or cell type.

It is contemplated that the tissue environment 12 may be a container suitable to encapsulate the tissue such as, for example, a test tube, beaker, or other suitable holding bottle. The container may be made from any suitable material such as glass, polycarbonate, plastic, etc. The container may be hermetically sealed or may be “breathable,” meaning that the container may permit the diffusion of gases, such as atmosphere, to permeate therethrough. Similarly, the container may be a flexible container such as a plastic bag or the like. The exact parameters for the container are not critical for operation of the apparatus 10 of the invention. As should be appreciated by those skilled in the art, there are a plethora of possible container options that may be employed without departing from the scope of the invention as described herein.

The environment 12 is contemplated to be connected to at least one fluid source and, in other contemplated embodiments, may be connected to a plethora of different fluid sources.

In the illustrated embodiment, the environment 12 is connected to a first fluid source 14 via a first fluid source line 16. In this embodiment, the first fluid source 14 is a source of nutrients for the tissue. The line 16 discharges the nutrient solution into the environment 12. As should be appreciated by those skilled in the art, the nutrient solution provides nutrients to the tissue to maintain the tissue in a viable condition for the duration of its encapsulation within the environment 12. The specific chemical composition of the nutrient solution is not critical to the functioning of the present invention.

While not illustrated, either the first fluid source 14 or the first fluid source line 16 may be provided with one or more control devices, including valves. The control devices may be controlled in any number of ways, as should be appreciated by those skilled in the art, to provide the nutrient solution in a manner sufficient to maintain the tissue in a viable condition. One control scheme contemplated for the system 10 is described in greater detail below in connection with the processor 30.

The tissue culture system 10 also includes a second fluid source 18 and a second fluid source line 20 that connects the second fluid source 18 to the environment 12. The second fluid provided from the second fluid source 18 is intended to include one or more biologically active agents to be perfused into the tissue. The precise combination of chemicals and pharmaceuticals included in the second fluid stream is not critical to operation of the present invention.

As with the first fluid source line 16, the second fluid source line 20 (and/or the second fluid source 18) may be provided with control devices to control the application of the biologically active agent(s) administered from the second fluid source 18.

With respect to the first fluid source 14 and the second fluid source 16, it is contemplated that a plurality of different sources may be connected to the environment. For example, the nutrient solution may be provided in the form of several different solutions, each of which are added to the environment 12 according to different timing protocols. The same may be said for the second fluid source 18 and the addition of biologically active agents. It may be desirable to add the biologically active agents from different sources at different times. Accordingly, multiple sources of the second fluid may be provided without departing from the scope of the invention.

It is noted that a stand-alone nutrient source 14 and a stand-alone source of biologically active agent(s) 18 are not required to practice the present invention. It is contemplated that the two sources 14, 18 may be combined into a single fluid source without departing from the scope of the invention.

In one contemplated embodiment, where the tissue is a small sampling of cells, it is anticipated that the tissue may be suspended in a solution within the environment 12 and that the first and second fluids will be discharged into the environment 12 as a part of the solution in which the tissue is suspended. Where a larger tissue sample is placed into the environment 12, the first and second solutions may be cannulated directly into the tissue via vessels or through direct injection, or with some other method. A combination of these two approaches also may be employed, as desired or required.

While the nutrient source 14 and the biological agent source 18 are described as fluids, it is contemplated that the two sources could provide dry (or partially dry) reagents to the environment 12, as desired. In such a circumstance, it is contemplated that fluids (i.e., saline and/or glucose) may be provided to the environment 12 via other means and that the dry reagents would be added directly to the fluid in which the tissue is suspended.

After the nutrient solution and the biologically active agent solution are introduced into the environment 12, the solutions are permitted to remain in the environment 12 for a predetermined period of time. This permits the biologically active agents, in particular, to become perfused within the tissue.

As should be appreciated by those skilled in the art, the tissue will metabolize the nutrients and the biologically active agents and excrete (and/or secrete) waste into the environment 12. The waste is then discharged through a waste line 22 to an effluent analyzer 24.

The timing of the discharge of the waste from the environment 12 to the effluent analyzer 24 may be controlled to occur as needed by the effluent analyzer 24. Alternatively, discharge of the effluent through the discharge line 22 may be controlled to occur at predetermined intervals. The exact timing and control over the discharge of effluent from the environment 12 to the effluent analyzer 24 is not critical to operation of the system 10 of the present invention.

The effluent analyzer 24 may be any of a number of different types of analyzers. For example, the effluent analyzer 24 may be a chromatograph or any other suitable device that permits analysis of the chemical constituents of the effluent (or waste) from the environment 12. It is anticipated that the effluent or waste will contain chemical signals that provide information concerning the development of diseases by the tissue. In the case of cancer research, it is anticipated that the tissue will excrete cancer precursors that may be detected by the effluent analyzer 24. Alternatively, the effluent analyzer 24 may be relied upon to detect changes in the amount of biologically active agent that has been metabolized by the tissue by evaluating the change in the amount of biologically active agent from the second source 18 to the waste line 22.

It is also contemplated that the effluent analyzer 24 may encompass a plurality of analyzers without departing from the scope of the invention. Those analyzers 24 may be connected, either in series or in parallel, with one another.

From the effluent analyzer 24, the effluent is passed through an effluent line 26 to an appropriate waste receptacle 28 for disposal. Alternatively, as discussed, the effluent may be passed from the effluent analyzer 24 to one or more additional analyzers or other equipment for further analysis and/or processing, as desired or required.

The tissue culture system 10 also includes a processor 30. The processor 30 is connected to the effluent analyzer 24 via a communication line 32. The processor 30 also is connected to the various other components, as illustrated. Specifically, the processor 30 connects to the first fluid source 14 via communication line 34 and to the second fluid source 18 via the communication line 36. A communication line 38 also connects the processor 30 to the environment 12.

In FIG. 1, the communication lines 32, 34, 36, 38 are shown as doted lines to indicate that the communication lines 32, 34, 36, 38 are electrical connections. This is intended to provide a visual distinction from the fluid lines 16, 20, 22, 26 that conduct fluid from one location to another. This convention is applied to the remaining figures for consistency. It is noted, however, that this convention should not be understood to be limiting of the invention. To the contrary, any suitable line may be employed in any of the instances illustrated in any of the figures. For example, it is possible that a fluid line may be used for communication between various components. Moreover, the communication lines could be wired or wireless communication lines, as should be appreciated by those skilled in the art.

With regard to the processor 30, it is contemplated that the processor 30 includes a central processing unit or CPU. Therefore, the processor 30 may be a personal computer, portable computing device, cellular phone, personal data assistant (“PDA”), or the like. The exact content and functionality of the processor 30 is not critical to the operation of the system 10, as should be appreciated by those skilled in the art.

In the illustrated embodiment, the processor 30 is capable of receiving electronic signals from the effluent analyzer 24, the first fluid source 14, the second fluid source 18, and the environment 12. In one contemplated example, the processor 30 may receive signals from the fluid sources 14, 18 concerning the amount of fluid in each source 14, 18. It is contemplated that the processor 30 will receive periodic updates concerning the level of fluids at each source 14, 18. Similarly, the processor 30 may receive information from the environment 12 concerning, among other types of data, the amount of fluid currently contained within the environment 12. Alternatively, the processor 30 may receive other information, i.e., temperature, needed to maintain the tissue in a viable condition.

It is also anticipated that the processor 30 will create and maintain an electronic log of the data provided by the components to which it is connected. In this capacity, the processor 30 serves as a repository of the information collected from the various components concerning any changes with respect to the tissue. In one contemplated embodiment, the processor 30 acts as a “flight recorder” by maintaining a continuous (or semi-continuous) log of the data fed to it from the various components of the system 10.

The processor also may execute one or more instructions based on the information received from the various components of the system 10 or from a user, such as a researcher. Upon execution of instruction in software code resident in the processor 30, the processor 30 may provide instructions to the first fluid source 14, the second fluid source 18, the environment 12, and/or the effluent analyzer 24. The instructions may encompass any of a number of commands. For example, the processor 30 may issue a command to the second fluid source 18 to supply a predetermined quantity of biologically active agent to the environment 12. Similarly, the processor 30 may issue a command to the first fluid source 14 to supply a predetermined quantity of nutrient solution to the environment 12. In addition, the processor 30 may issue a command to the effluent analyzer 24 to initiate a particular type of analysis on the effluent from the environment 12. A multitude of other types of commands may be issued from the processor, as would be understood by those skilled in the art.

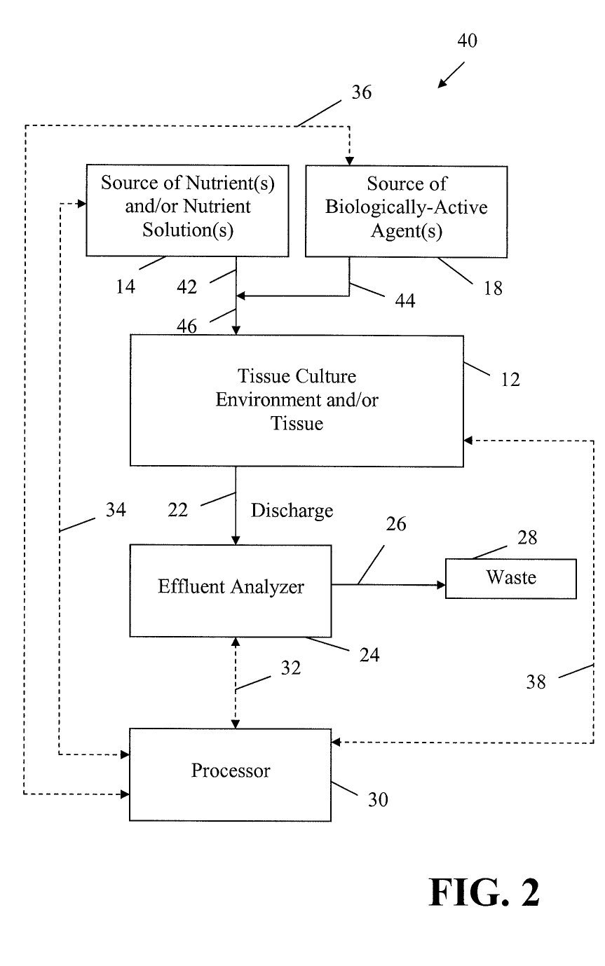

FIG. 2 provides a schematic of a second embodiment of a tissue culture system 40 according to the present invention. In FIG. 2, the system 40 differs from the system 30 in that the fluid lines 16, 20 have been replaced with fluid lines 42, 44, 46.

In this second embodiment, the separate lines 16, 20 from the first system 10 have been combined so that the fluid line 44 from the source 18 of biologically active agent(s) discharges the biologically active agent(s) into a common fluid line 46. The line 42 extending from the nutrient source 14 also discharges nutrient solution into the same, common fluid line 46. In this embodiment, therefore, there is only one outlet from the sources 14, 18 into the environment 12. In all other respects, the system 40 is similar to the system 10. Accordingly, the discussion of the components in the system 10 applies equally to the system 40, as should be appreciated by those skilled in the art.

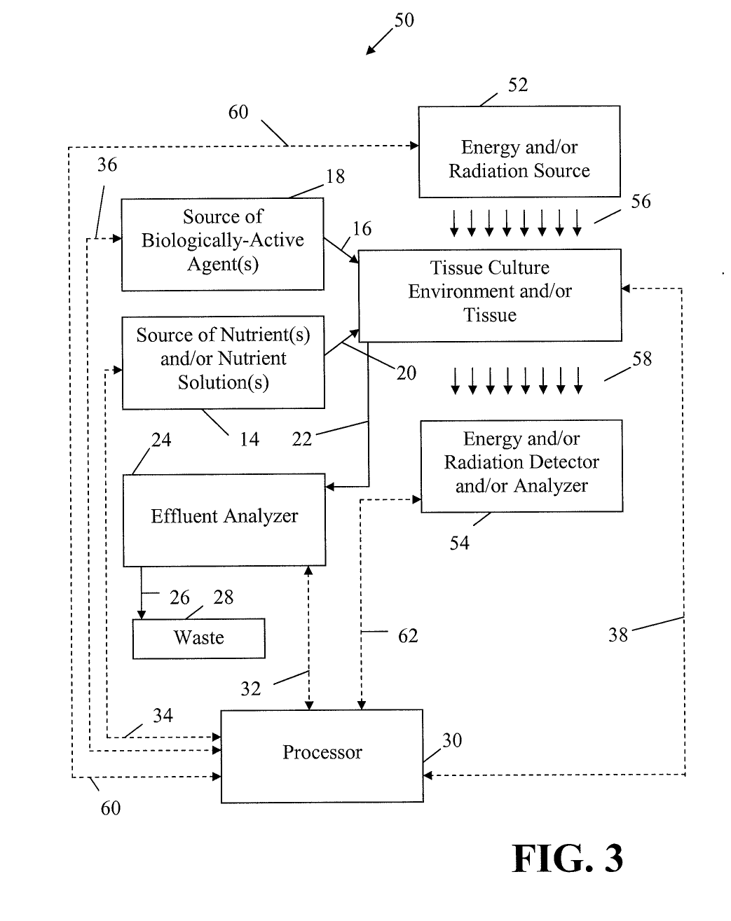

FIG. 3 provides a schematic illustration of a variation of the system 10 illustrated in FIG. 1. FIG. 3 presents a tissue culture system 50.

To the components in the tissue culture system 10, tissue culture system 50 adds an energy source 52 and an energy detector and/or analyzer 54. Since the energy generated by the energy source 50 and the energy received by the energy detector/analyzer 54 are contemplated to be electromagnetic radiation, these components are also referred to as radiation source (or radiation generator) 52 and radiation detector/analyzer 54 herein. It is contemplated, however, that electromagnetic radiation is not the only type of energy generated by the energy source 52 or received by the energy detector/analyzer 54. Other types of energy include electrical energy and magnetic energy, as in the case of a magnetic resonance imager (or “MRI”).

If the energy is electromagnetic radiation, the radiation source 52 generates radiation 56 that is introduced to the tissue in the environment 12. Exigent radiation 58 is then directed or detected by the radiation detector 54. By analyzing the exigent radiation 58, the conditions of the tissue in the environment may be analyzed to evaluate a change in condition as a result of the addition of biologically active agent(s) from the source 18.

The radiation 56 generated by the radiation generator 52 may be any of a variety of different types. For example, the radiation generator 52 may be an x-ray source to provide x-rays for studying, over time, potential growth of cancerous tissue within the tissue sample in the environment 12.

Alternatively, the radiation generator 52 may emit visible light. In this contemplated embodiment, the light would be directed through the tissue sample to illuminate any cancerous materials contained within the tissue sample.

In still another example, as discussed above, the energy generator 52 may be a magnetic coil that generates a magnetic field. In this contemplated example, the energy generator 52 and the energy detector 54 cooperatively act as a magnetic resonance imager (“MRI”). As should be appreciated by those skilled in the art, any number of other types of radiation and/or energy 56 may be applied to the tissue for analytical purposes.

In still another contemplated embodiment, the radiation generator 52 may generate radiation 56 that may be applied to test one or more therapies for causing the cancerous tissue to enter a remissive state. For example, the system 50 may be configured to apply low dose gamma radiation 56 to the tissue to analyze the efficacy of the radiation 56 on the tissue within the environment 52.

In still another embodiment, the radiation generator may emit ultrasound waves.

Other types of radiation also may be tested via the system 50, as should be appreciated by those skilled in the art.

With continued reference to FIG. 3, the processor 30 includes a communication line 60 to the energy generator 52 and a communication line 62 to the energy detector/analyzer 54. The communication lines 60, 62 permit communication between the processor 30, the energy generator 52, and the energy detector/analyzer 54. As with other components of the system 50, the processor 30 may receive data from the energy source 52 and the energy detector/analyzer 54. The processor 30 also may issue operating instructions to the same components.

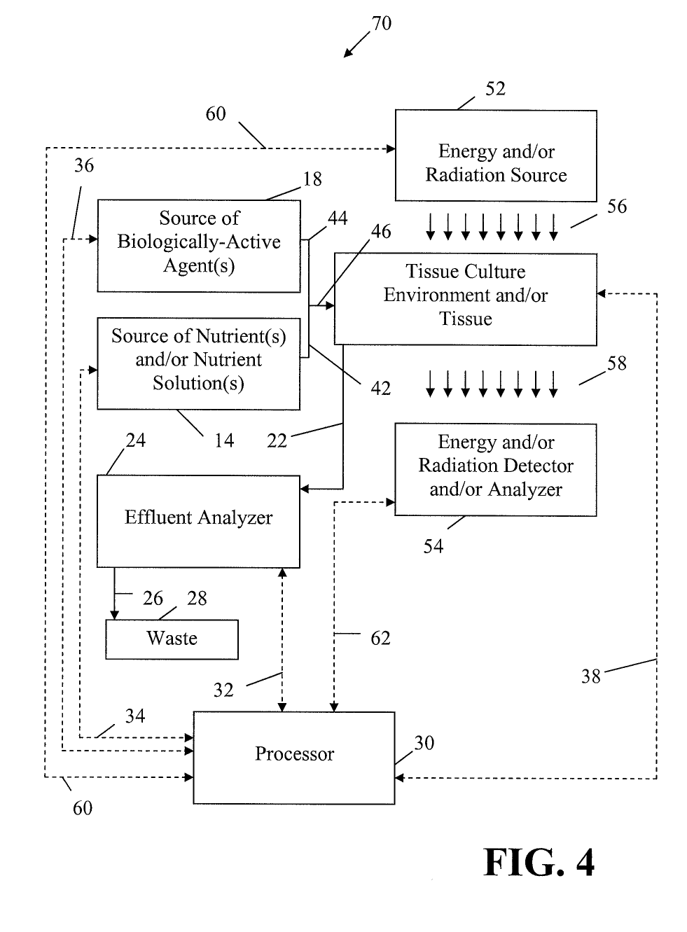

FIG. 4 is a variation of the system 40 illustrated in FIG. 2. Here, a schematic illustration of provided for a tissue culture system 70. In this embodiment, the energy source 52 and the energy detector/analyzer 54 have been added to the system 40 illustrated in FIG. 2.

Before discussing the method of the present invention, additional details are provided with respect to the various systems 10, 40, 50, 70 described above.

As noted above, the systems 50, 70 include an energy source 52 and an energy detector/analyzer 54. These components are contemplated to permit a modification of the systems 50, 70 so that non-invasive imaging of the vital isolated breast may be performed. These imaging methods may include modalities typically applied to patients, such as x-ray mammography or ultrasound, which do not require the administration of exogenous contrast materials. The imaging methods may also include modalities that require prior administration of exogenous materials, for example positron emission mammography (which requires administration of radioactive ligands) or contrast-enhanced magnetic resonance imaging (which typically requires administration of chemicals containing paramagnetic materials such as Gadolinium). The invention provides for administration of such contrast materials via some of the cannulated vessels, mimicking systemic administration. The invention provides for local administration of contrast materials, via needles introduced into the breast, which may be targeted by the imaging methods described above.

It is also contemplated that the systems 10, 40, 50, 70 may be modified to permit sampling of the isolated breast, such as by fine-needle aspiration or core biopsy. The locations for sampling may be provided by the imaging methods described above. The sampling evaluation may include biochemical assays as well as pathological examinations (e.g., histology). The sampling may be done automatically or via more traditional, manual means, as should be appreciated by those skilled in the art.

As noted above, the tissue placed in the environment 12 may be a complete organ that has been cannulated to receive nutrients and biologically active agents. In such an arrangement, it is contemplated for the administration of biologically active agents to include pharmaceuticals that are administered via some of the cannulated vessels, mimicking systemic administration, or via needles or catheters locally, at locations which may be targeted with the assistance of imaging methods described above.

It is contemplated that other biologically active agents and/or interventions may be delivered physically, for example, by external or internal sources of ionizing radiation.

One embodiment of the invention contemplates that healthy tissue will be subjected to perfusion of various hormones and biological stimulants. As discussed above, the tissue culture is located within the environment 12 to permit continuous or semi-continuous monitoring of the tissue. In this contemplated embodiment, the healthy tissue may be treated with biologically active agents to initiate the formation of a cancer, thereby permitting study of carcinogenesis.

As discussed above, the tissue may be maintained in the environment 12 that includes or adjacent to a magnetic resonance imager (“MRI”), which is one contemplated variation for the energy source 52. As discussed above, it is contemplated that periodic operation of the MRI will be initiated, automatically or semi-automatically, to establish data points with respect to the tissue's response to various stimuli, i.e., the biologically active agents. The data provided to the processor 30 may then be analyzed to provide insight into one or more mechanisms for the formation of cancer cells (or diseased cells) within a living organism.

As discussed above, there are a wide variety of monitoring devices that are contemplated to be used with (or as a part of) the systems 10, 40, 50, 70. For example, an optical device is one type of device that is contemplated to be employed. Due to the sheer number of different types of observation devices that may be employed, an exhaustive list is not provided. While not listed, the invention is intended to encompass any observation device that may be employed to capture data with respect to living tissue.

As noted above, an MRI is contemplated as one of the observation devices (i.e., detector 54) that may be employed as a part of the apparatus and the method of the invention. In this example, the researcher's hypothesis may include the application of a humeral factor (e.g., insulin-like growth factor) to a tissue culture that results in an increase in the mass of the tissue culture. For the purpose of this application, such humeral factors are designated as “growth hormones,” although the class of such materials encompasses many materials other than human growth hormone.

To test this hypothesis, the researcher may obtain a suitable tissue culture and place that tissue culture in the environment 12 to sustain the viability of the tissue culture. Details of the environment 12 are discussed above. The environment 12, in turn, is connected to one or more sources 18 of chemical substances (i.e., biologically active agents), such as the growth hormone being tested. The hormone may be introduced to the environment 12 and, thereby, to the tissue culture.

Once the tissue is exposed to the chemical substance (i.e., the growth hormone), the tissue may react. In this example, an MRI is positioned near or around the tissue sample. The MRI, a least in one contemplated embodiment, is activated automatically to collect data on a regular and sustained basis. Growth hormone is then injected into the tissue culture on a regular basis. The MRI is programmed to assess new vessel growth of the tissue sample at predetermined time intervals. In this manner, the effect of the growth hormone on the tissue may be correlated with the application (or perfusion) of the growth hormone into the tissue sample.

This same basic approach may be applied to any interaction between a chemical stimulus and any cell. In the context of the invention described herein, the starting tissue is anticipated to be healthy (i.e., non-diseased tissue). The healthy tissue is then subjected to biochemical stimuli (or stresses) to transform the tissue into diseased (i.e., cancerous) tissue. By this process, it is anticipated that it will be possible to develop more thorough understandings of the biochemical processes in the human body that result in cancerous cells, among other afflictions.

As discussed above, the environment 12 may be monitored automatically or semi-automatically. In an automatic mode, the detection device (i.e., the MRI) may be set to measure specific parameters on a repetitive basis. For example, data may be collected one per minute, per hour, per day, etc. In a semi-automatic mode, the measurements may be triggered by input received from the researcher or by other parameters and conditions from others of the components in the system 10, 40, 50, 70.

As discussed above, in another contemplated embodiment, the detection device 24 may be connected to an effluent port 22 connected to the environment 12 chamber containing the tissue culture. In this embodiment, the detector 24 may be a chromatograph or similar device that detects the chemical substances excreted by the tissue culture. With such a construction, it is possible to determine how the tissue culture is processing the inputted chemical substances by examining the materials metabolized by the tissue culture.

In further embodiments, several detection devices 24, 54 of varying types may be employed in connection with the environment chamber 12 to detect several parameters concerning the tissue culture.

With this basic framework in mind, it is contemplated that the invention may be used in different contexts: (1) as a tool for the development of drugs for therapeutic treatments, and (2) as a tool for the development of non-drug interventions (e.g., ionizing radiation) for therapeutic treatments, and (3) as a tool for the development of diagnostic methods (e.g., non-invasive imaging, chemical assays).

With respect to the first use of the invention, it is contemplated that the apparatus and method of the invention will assist researchers to develop new drugs for the treatment of various diseases, including cancers, such as breast cancer. As a tool for the development of drugs, the apparatus and the method of the invention may be employed to study the reaction of tissues to one or more chemical substances. Depending upon the reaction by the tissues in the isolated breast model, new drugs or new combinations or new doses of known drugs, may be identified and/or developed for later clinical trials, for example.

With respect to the second use of the invention, it is anticipated that the apparatus and method of the invention will assist researchers to develop new radiation therapies for the treatment of various diseases, including cancers, such as breast cancer.

With respect to the third use of the invention, it is anticipated that the analysis devices and tools used to monitor and take data with respect to the tissues of the isolated breast model may be applied to intact breasts in human beings. For example, the combination of detection devices, whether invasive or not, may be connected to a human subject to evaluate the biological processes existing in tissues in vivo.

In this context, it is noted that an MRI may be employed to analyze a tissue culture by examining the tissue culture's response to a particular chemical stimulus in vivo.

Other variations of the invention also are contemplated to fall within the scope of the invention, as should be apparent to those skilled in the art.

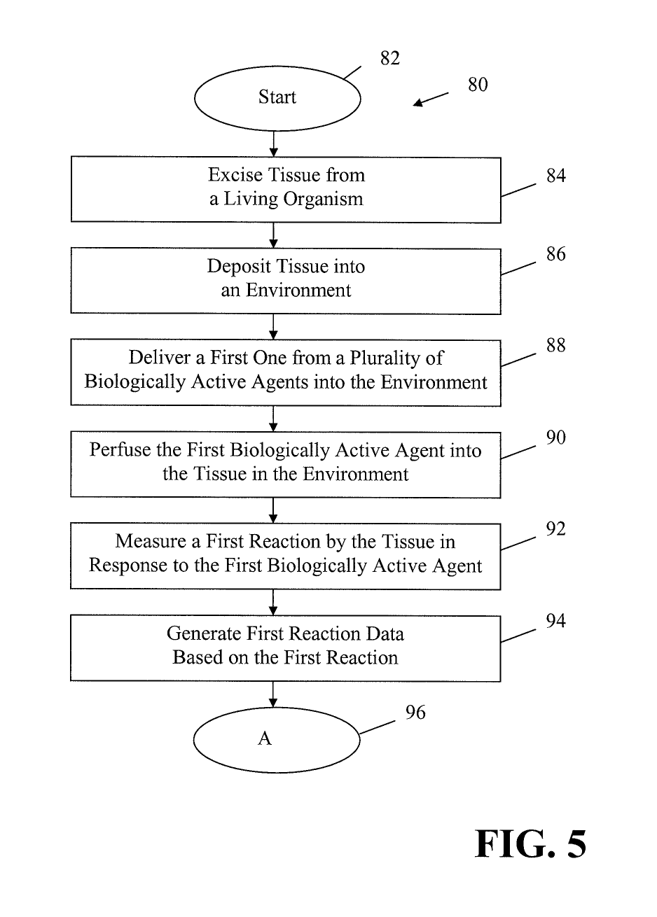

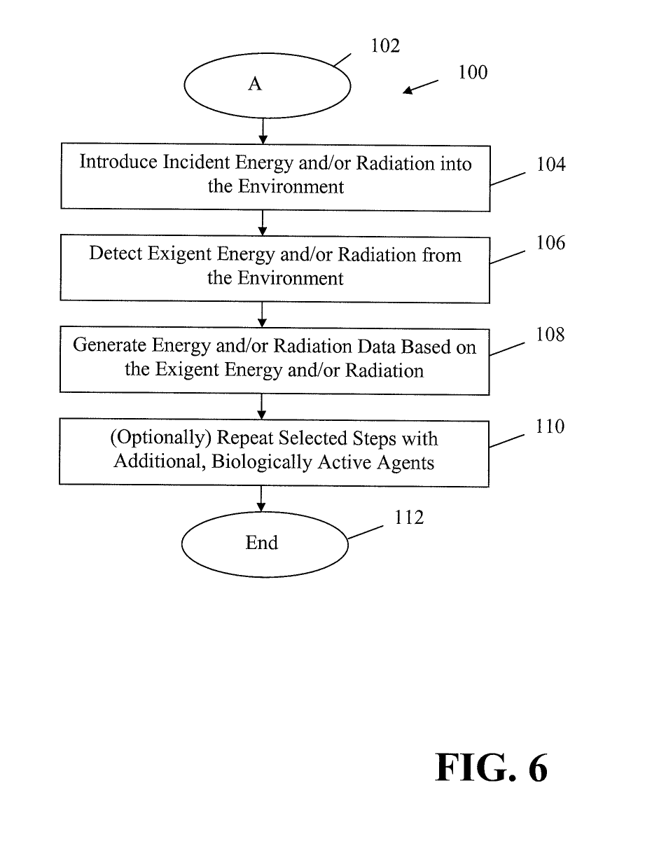

Reference is now made to FIGS. 5 and 6, which illustrate a flow chart for one method contemplated by the present invention.

The method 80 begins at start 82. The method 80 first requires the excising of tissue from a living organism, which is illustrated at 84. It is noted, however, that tissue also may be removed from a recently-deceased organism as well. The term “living organism” is not meant to convey that the organism must be alive at the time of excision of the tissue, only that the organism was at one time alive and that viable tissue is removable therefrom.

After the tissue is excised at 84, the tissue is deposited into the environment 12 at 86. Details of the environment 12 are provided above. The environment 12 is intended to maintain the tissue in a viable condition for a predetermined period of time sufficient to conduct a study of the tissue. It is contemplated, therefore, that the environment 12 will maintain the tissue in a viable condition for a period of at least one week. Of course a longer period or a shorter period also is intended to fall within the scope of the invention.

Once the tissue has been deposited into the environment 12 at 86, at least one biologically active agent is delivered to the environment 12 at 88. As noted above, the biologically active agent may be any type of agent that elicits a response from the tissue.

After 88, the biologically active agent is perfused into the tissue in the environment 12 at 90. As above, the biologically active agent may encompass a plurality of biologically active agents and may not be limited solely to a single agent.

Then, at 92, a reaction by the tissue to the biologically active agent is measured. As noted above, the measurement may be accomplished by analyzing the effluent removed from the environment.

At 94, reaction data is generated based on the reaction of the tissue to the biologically active agent.

The method transitions from FIG. 5 to FIG. 6 via the connectors 96, 102.

FIG. 6 illustrates an optional portion 100 of the method of the invention. In this portion of the method, at 104, the tissue is exposed to energy and/or radiation 56, as discussed above.

At 106, exigent energy and/or radiation 58 is detected.

At 108, data is generated based on the exigent energy and/or radiation 58. This step constitutes the end of the method 100, at least according to one contemplated embodiment of the invention.

At 110, selected steps are repeated, either automatically or semi-automatically, as discussed above.

As noted above, the present invention encompasses various apparatuses, systems, and methods that are described in connection with the embodiments described herein. The invention, however, is not intended to be limited solely to the embodiments discussed. To the contrary, the invention is intended to encompass variations and equivalents, as should be appreciated by those skilled in the art.

What is claimed:

1. An apparatus for evaluating the effects of stimuli on tissue, comprising:
an environment for holding at least a sample of the tissue from a living person;
a first source connected to the environment for delivering nutrients to the tissue to maintain the tissue in a viable condition for more than one week;
a second source connected to the environment for delivering a biologically active agent to the tissue, wherein the biologically active agent is capable of inducing carcinogenesis in the tissue; and
an effluent outlet connected to the environment to remove effluent from the environment.

2. The apparatus of claim 1, further comprising:
an effluent analyzer connected to the effluent outlet to analyze the content of the effluent and generate effluent data based thereon; and
a processor connected to the effluent analyzer to collect and store the effluent data at predetermined time intervals, thereby permitting capture of information concerning a reaction by the tissue to the biologically active agent.

3. The apparatus of claim 1, further comprising:
a processor connected to the first and second sources to control the timing of delivery of at least one of the nutrients and biologically active agents at predetermined time intervals.

4. The apparatus of claim 1, wherein the tissue is at least one of a resected, human organ, a human, female breast, healthy human tissue, and precancerous, human tissue.

5. The apparatus of claim 1, wherein the first source and the second source are connected independently to the environment.

6. The apparatus of claim 1, wherein the first source and the second source are connected to the environment via a common connection.

7. The apparatus of claim 1, further comprising:
a radiation source disposed adjacent to the environment for directing incident radiation into the environment, at the tissue, thereby generating exigent radiation; and
a radiation detector disposed adjacent to the environment to measure the exigent radiation from the environment and to generate radiation data based thereon.

8. The apparatus of claim 7, further comprising:
a processor connected to the radiation source and the radiation detector, wherein the processor controls at least the introduction of radiation to the environment at predetermined intervals.

9. The apparatus of claim 7, wherein the radiation source generates at least one of electromagnetic radiation, x-ray radiation, gamma radiation, thermal radiation, visible light, and ultraviolet light.

10. The apparatus of claim 7, wherein the radiation detector further comprises an imager that generates a composite image based upon the exigent radiation.

11. A method for evaluating the effects of stimuli on tissue, comprising:
introducing the tissue, removed from a living person, into an environment capable of maintaining the tissue in a viable condition for at least one week;
delivering at least a first one from a variety of biologically active agents into the environment from a first source;
perfusing the first biologically active agent into the tissue with the intent of inducing carcinogenesis within the tissue;
measuring a first reaction by the tissue in response to the first biologically active agent; and
generating first reaction data representative of the first tissue response.

12. The method of claim 11, further comprising:
delivering at least a second biologically active agent into the environment from a second source;
perfusing the second biologically active agents into the tissue within the environment;
measuring a second reaction by the tissue in response to the second biologically active agent; and
generating second reaction data representative of the second tissue response.

13. The method of claim 11, further comprising:
introducing incident radiation into the environment at the tissue, thereby generating exigent radiation; and
detecting exigent radiation from the environment to generate radiation data based thereon.

14. The method of claim 11, further comprising:
providing the first reaction data to a processor at least for storing the first reaction data.

15. The method of claim 14, wherein, via the processor, the first reaction data assists with control of the delivery of the first biologically active agent into the environment.

16. The method of claim 13, further comprising:
providing the radiation data to a processor at least for storing the radiation data.

17. The method of claim 16, wherein the processor controls the delivery of incident radiation into the environment at least in part based upon the radiation data.

18. The method of claim 11, wherein the tissue is at least one of a resected, human organ, a human, female breast, healthy human tissue, and precancerous, human tissue.

19. The method of claim 18, wherein the tissue is removed from a human, female breast as part of a mammoplasty procedure.

20. The method of claim 19, wherein the tissue is removed from a breast as part of a therapeutic procedure.
